(19) United States
(12) Patent Application Publication
(10) Pub. No.: US 2009/0132008 A1
(43) Pub. Date: May 21, 2009

(54) MEDICAL APPARATUS AND SYSTEM

(76) Inventors: Tomas Snitting, Stockholm (SE); Viktor Skoog, Hasselby (SE); Hans Abrahamson, Stockholm (SE)

Correspondence Address: SCHIFF HARDIN, LLP;PATENT DEPARTMENT, 6600 SEARS TOWER, CHICAGO, IL, 60606-6473, US

(21) Appl. No.: 11/916,315

(22) PCT Filed: Jun. 3, 2005
(86) PCT No.: PCT/SE2005/000854

§ 371 Date: Jul. 16, 2008

(51) Int. Cl. A61N 1/08

(52) U.S. Cl. 607/60

(57) ABSTRACT

A medical system includes an implantable medical device configured for implantation in a living subject to interact with the subject, an extracorporeal device having a processor that processes information such as programming instructions for the implantable medical device or monitoring data received from the implantable medical device, and at least two antenna devices located at respectively different locations. The implantable medical device communicates with at least one of the two antenna devices, and the extracorporeal device also communicates with the at least two antenna devices to exchange the aforementioned information with the implantable medical device via at least one of the two antenna devices. The at least two antenna devices are physically separated from the extracorporeal device and the extracorporeal device communicates with each of the at least two antenna devices via a communication link that allows the extracorporeal device to be freely moved relative to the at least two antenna devices. The communication link can be a hard-wired communication link or a wireless communication link.

### BACKGROUND OF THE INVENTION

1. Field of the Invention

The present invention relates to the field of telemetry, and in particular to a medical apparatus for programming and/or monitoring an implantable medical device over a radio-based wireless network, and such a system.

2. Description of the Prior Art

Telemetry is a generic term for techniques for conveying measuring data from one point to another, usually by means of radio. In particular, within the medical field telemetry systems are generally used for enabling radio-frequency (RF) communication between an implantable medical device (IMD) such as a pacemaker, and an external monitoring device. The frequency spectrum used for wireless communications between implanted medical devices and external equipment is about 400 MHz, but for wireless medical telemetry services in general several frequency bands may be used. Within a medical telemetry system crucial physiologic data is transmitted, and it is critical to ensure that data is not lost or delayed. Medical telemetry is a low-power radio system, and although relatively short distances are usually employed within such systems, there may nevertheless arise a need for considering reception aspects. One such consideration is related to the fact that electromagnetic fields emitted in a room will give rise to standing wave patterns. The energy that a receiver will be able to receive is varying as a function of the position in the room. Using multiple antennas, resulting in so called spatial diversity, may minimize the effects of this.

U.S. Pat. No. 6,167,312 discloses such a device for use in communication with an implantable medical device. The device is provided with a spatial diversity antenna array including at least one antenna permanently and fixedly mounted to the housing of a monitor or programmer, and an additional antenna removably mounted to the housing.

This known telemetry system, although suggesting the use of spatial diversity in order to facilitate the reception of signals from an implantable device and also the transmission of signals to the implanted device, still has several drawbacks regarding the signaling. For example, as mentioned above, the system comprises a removable antenna, but the use of it entails the physician having to move the antenna around until an acceptable reception is obtained. Therefore, should there arise a need to move a patient from one place to another, for example from an examination room to an X-ray examination room, the tedious reception/transmission optimization would have to be performed once more. Thus, the apparatus described requires the physician operating it to perform a kind of an antenna reception optimization, which is a time-consuming and also unreliable method. Further, the range of said removable antenna is limited, and dependent upon the length of a coiled cord by means of which the removable antenna is coupled to a transceiver within the programmer.

Furthermore, such a programmer is relatively expensive, and it would be desirable and convenient to be able to easily move the programmer, for example between different wards in a hospital, with retained communication quality, to thereby avoid having to buy several costly programmers.

There is thus a need in a telemetry system, for improved two-way communication of signals between a monitoring device and an implantable medical device, both forming parts of a medical system for programming and/or monitoring the implantable medical device over a radio-based wireless network. In particular, it would be desirable to provide a reliable communication, which overcomes the aforementioned shortcomings of known systems and devices.

### SUMMARY OF THE INVENTION

An object of the present invention is to provide reliable radio communication within a telemetry system, the communication being easily and conveniently optimized, eliminating or at least reducing the risk of a communication failure between an implantable medical device and a monitoring device due to fading and/or a low signal strength.

It is a further object of the present invention to provide a medical apparatus and system, by means of which spatial diversity is achieved.

These objects are achieved in accordance with the present invention by a medical apparatus having a monitoring device and at least two antenna devices, the medical apparatus enabling programming and/or monitoring of an implantable medical device over a radio-based wireless network. The at least two antenna devices in the system are provided as separate, stand-alone units, i.e. not forming part of the programmer or monitoring device. Thus it is possible to place the antenna devices in an optimal way, preferably at stationary locations, such as for example wall and/or ceiling mounted. The antennas may be placed in each room, or area of use, in which telemetry is utilized, for example an X-ray room, examination room or operating room, or even in the equipment utilized. Since, in accordance with the present invention, the distance between a patient and the programmer no longer is a consideration with regard to signal reception, the programmer may be easily moved from one place to another without thereby affecting the signal quality. The placement of the antennas may also be optimized in advance, in consideration of where in the respective rooms the patient usually is located.

In accordance with an embodiment of the present invention the medical apparatus further has a control unit provided for measuring a signal quality parameter of signals received from the implantable medical device by each of the antenna devices. Thereafter one of the antenna devices is selected for subsequent reception or transmission of signals from the implantable medical device depending on the measured signal quality parameter of the received signals. Spatial diversity is thereby ensured, and the antenna giving the best reception may be chosen and a reliable communication is provided.

In accordance with another embodiment of the present invention the signal quality parameter is any of RSSI, BER, or C/N ratio. A flexibility in how to chose a suitable antenna, i.e. in dependence on an optional parameter, is thereby provided. These parameters are commonly known and often used in assessing signal quality, thus enabling the use of well established, easily obtainable algorithms.

In accordance with yet another embodiment of the present invention the control unit is utilized for measuring a signal quality parameter of signals received from the implantable medical device by each of the antenna devices at regular intervals, or continuously. It is thereby possible to rapidly detect a deteriorated signal quality and switch to an antenna having a better signal reception.

In accordance with yet another embodiment of the present invention the control unit is connected between the programmer or monitoring device and the antenna devices. In an alternative embodiment, the control unit is an integral part of the programmer. In yet a further embodiment, the control unit is provided as an integral part of either one of the antenna devices. This provides a modular structure, giving a great design flexibility, and enabling custom-made solutions.

In accordance with yet another embodiment of the present invention the communication links between the programmer or monitoring device and the control unit, and between the control unit and the antenna devices, may be via wire, e.g. an USB connection, or wirelessly, e.g. via Bluetooth. This again adds to the design flexibility.

In accordance with yet another embodiment of the present invention each of the antenna devices comprises a radio transceiver unit. In another embodiment, only one transceiver unit is provided, preferably centrally located in a room, or other area of use. Utilizing several radio transceiver units provides an additional security, but if a less expensive solution is desired, a fewer number of radio transceiver units may be provided.

In accordance with still another embodiment of the present invention each of the antenna devices are fixedly mounted, for example in a ceiling or to a wall. Thereby the antenna devices may be more or less permanently placed at locations considered to be the best in view of reception/transmission from and to an implantable medical device. The reception/transmission may be optimized in advance, in dependence of an expected location in a room of the patient wearing the implantable medical device.

In accordance with still another embodiment of the present invention each of the antenna devices comprises a conductive radiating antenna element, and these conductive radiating antenna elements are adapted to emit and receive radio waves having essentially parallel polarization Thereby spatial diversity is provided independently of polarization diversity.

In accordance with still another embodiment of the present invention each of the antenna devices comprises at least one conductive radiating antenna element capable of emitting and receiving radio waves of orthogonal polarizations. If at least two conductive radiating antenna elements are provided in each antenna device they should be operatively provided adjacent to each other at a single location in space.

In accordance with still another embodiment of the invention the programmer or monitoring device is portable, and is in particular a hand held device. In accordance with the present invention, the antenna devices are not physically part of the programmer or monitoring device, which would, considering the frequencies in question, require a certain, non-portable size of the programmer in order to accommodate the fastening of several antennas to it. The size of the programmer may therefore be reduced in accordance with the invention. A user may thereby easily bring the programmer along, should such need arise. In another embodiment, the programmer or monitoring device is arranged on a movable rack such as a roller table or the like, whereby the present invention may be utilized also in connection with currently used programmer or monitoring devices, providing a solution that is easy to implement with existing programmers.

The present invention is also related to such a system, in accordance with which advantages corresponding to the above described are achieved.

### BRIEF DESCRIPTION OF THE DRAWINGS

FIG. 1 is a schematic block diagram of an exemplary embodiment of a medical apparatus in accordance with the present invention.

FIG. 2 is a schematic block diagram of another exemplary embodiment of a medical apparatus in accordance with the present invention.

FIG. 3 shows a system in accordance with the present invention.

### DESCRIPTION OF THE PREFERRED EMBODIMENTS

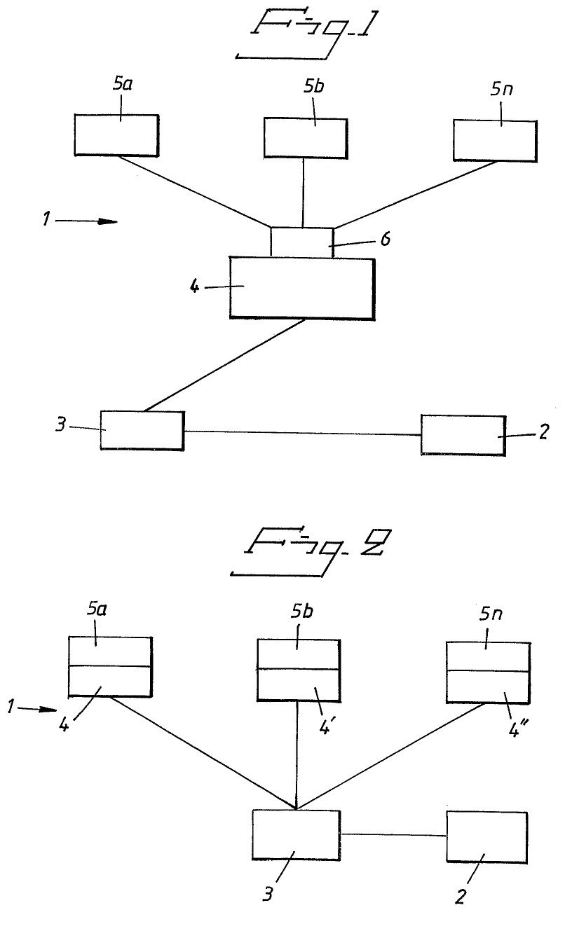

In the following description the same reference numerals will be used for equivalent or similar elements throughout the drawings. With reference first to FIG. 1, a schematic layout depicting an exemplary medical apparatus in accordance with the invention is shown. A medical apparatus 1 for programming and/or monitoring a patient related device 7 shown in FIG. 3, for example an implantable medical device, over a radio-based wireless network comprises a programmer or monitoring device 2, hereinafter referred to as a programmer 2. The programmer 2 is provided with input and/or output means for transmitting programming instructions to an implantable medical device, and/or for outputting monitoring information patient related data, for example for display on a screen, thereby enabling a physician to easily see such patient related data received from the implantable medical device.

A control unit 3, for example a microcontroller, is connected to the programmer 2, via wired standards, for example via USB (Universal Serial Bus), or via some wireless protocols. Bluetooth is such an exemplary, preferred wireless protocol, being an open-standard protocol. Using an open-standard protocol allows interoperability among devices from different manufacturers, which may be very advantageous in some cases. For example, utilizing Bluetooth standard for communication between the programmer 2 and antenna devices may permit the use of programmers from different producers, without also necessitating antenna device changes, which is particularly advantageous if the antenna devices are wall mounted or in some other manner more permanently mounted. In the embodiment shown in FIG. 1, the control unit 3 and the programmer 2 are shown as separate parts, but it is possible to, in an alternative embodiment, make the control unit 3 an integrated part of the programmer 2.

The control unit 3 is connected to at least one radio frequency circuitry unit 4, hereinafter called transceiver unit, via a digital link such as SPI (Serial Peripheral Interface), USB, Bluetooth or the like. The control unit 3 controls the one or more transceiver units 4. The transceiver unit 4 embodies conventional radio frequency circuitry, such as, for example, a duplexer, connected to a transmitter section and a receiver section, microcontroller, a wakeup transmitter, switches, low noise amplifiers (LNA), power amplifiers, AGC (Automatic Gain Control), power detectors and filters. The transceiver unit may also be an integral part of the contol unit 3.

The medical apparatus 1 further includes at least two antenna devices 5a, 5b, . . . , 5n operatively provided at different locations, that is, they are provided as separate, stand alone units, i.e. not forming part of the programmer 2 as in the prior art. The programmer 2 is connected, via a control unit 3 and transceiver unit 4, to the antenna devices 5a, 5b, . . . , 5n and is provided for transmitting signals to and receiving signals from an implantable medical device via either one of the antenna devices 5a, 5b, . . . , 5n. The connection between the antenna devices 5a, 5b, . . . , 5n and the programmer 2 is a wired connection, e.g. an USB connection, or a wireless connection, e.g. via Bluetooth. Thereby movements of the programmer 2 are enabled, while the antenna devices 5a, 5b, . . . , 5n are kept still, for example being permanently mounted to a wall or the like. By means of the invention it is possible to place the antenna devices 5a, 5b, . . . 5n in an optimal way, preferably at stationary locations, such as for example wall mounted. The antenna devices 5a, 5b, . . . 5n may be placed in each room, or area of use, in which telemetry is utilized, for example an X-ray room, examination room or operating room, or even in the equipment utilized. Since, in accordance with the present invention, the distance between a patient and the programmer 2 is not a consideration with regard to signal reception anymore, the programmer 2 may be easily moved from one place to another without the signal quality being affected. The placement of the antenna devices 5a, 5b, . . . 5n may also be optimized in advance, in consideration of where in a respective room the patient usually is located. For example, in an X-ray room the patient is most likely placed at a certain location known in advance, and the antenna devices 5a, 5b, . . . , 5n may be placed so as to optimize the reception/transmission in relation to this location. In the embodiment shown in FIG. 1 the antenna devices 5a, 5b, . . . , 5n consist only of antenna elements, i.e. electrically conductive and radiating structures for transmitting and receiving radio frequency signals. These antenna elements can have any desired and appropriate shape, such as for example strip shape, cross shape or star shape. In the figure three such antenna elements are shown, but it is understood that any suitable number of antenna elements may be used. Further, each antenna device 5a, 5b, . . . , 5n may comprise means for enabling polarization diversity, for example by providing the antenna device 5a, . . . , 5b, . . . 5n with conductive structures for emitting and/or receiving radiation of different polarizations. In this regard, reference is made to the pending International application, no. PCT/SE2004/000832, entitled “Medical transceiver device and method”, having the same applicant as the present application. Its disclosure is incorporated herein by reference.

The antenna devices 5a, 5b, . . . , 5n are connected to the transceiver unit 4, the transceiver unit 4 being controlled by the control unit 3. A switch device 6, switchable between using one or more of the different antenna devices 5a, 5b, . . . , 5n is also included. When utilizing spatial diversity, advantage is taken of the different paths of a wave propagation in a reflective environment, and the antenna device 5a, 5b, . . . , 5n giving the best reception at any time may be utilized. In accordance with the invention thus, the antenna device 5a, 5b, . . . , 5n giving the best communication link, as determined in a suitable way, is chosen for communication between the programmer 2 and an implantable medical device. The control unit 3 includes circuitry for measuring characteristics of the radio frequency signals as received by the antenna devices 5a, 5b, . . . , 5n. Depending on a suitable signal quality indicator one of the antenna devices 5a, 5b, . . . , 5n is chosen for the subsequent communication. The signal quality indicator or parameter may for example be one of: signal strength, bit error rate (BER), carrier-to-noise (C/N) ratio, carrier-to-interference (C/I) ratio or received signal strength indicators (RSSI). In an alternative embodiment, requiring more signal processing, the signals from two or more of the different antenna devices 5a, 5b, . . . , 5n are combined, i.e. the different paths are put in phase and then added. It is possible to perform regular polling of all antenna devices 5a, 5b, . . . , 5n or transceiver units 4 in order to keep track of the signal quality at different places in the room. In an alternative embodiment, the control unit is set on continuous listening of the antenna devices 5a, 5b, . . . , 5n or transceiver units 4.

Alternatively, the medical apparatus 1, and in particular the control unit 3 thereof, receives from an implantable medical device a measure of a signal quality parameter of signals as received by the implantable medical device, wherein the signals received by the implantable medical device are signals as transmitted from the medical apparatus to the implantable medical device after having been distorted by a transmission medium, i.e. the air interface between the respective antenna devices. The signal strength and the phase of the signals thereafter transmitted may be altered in dependence on the signal quality parameter of the signals as received by the implantable medical device.

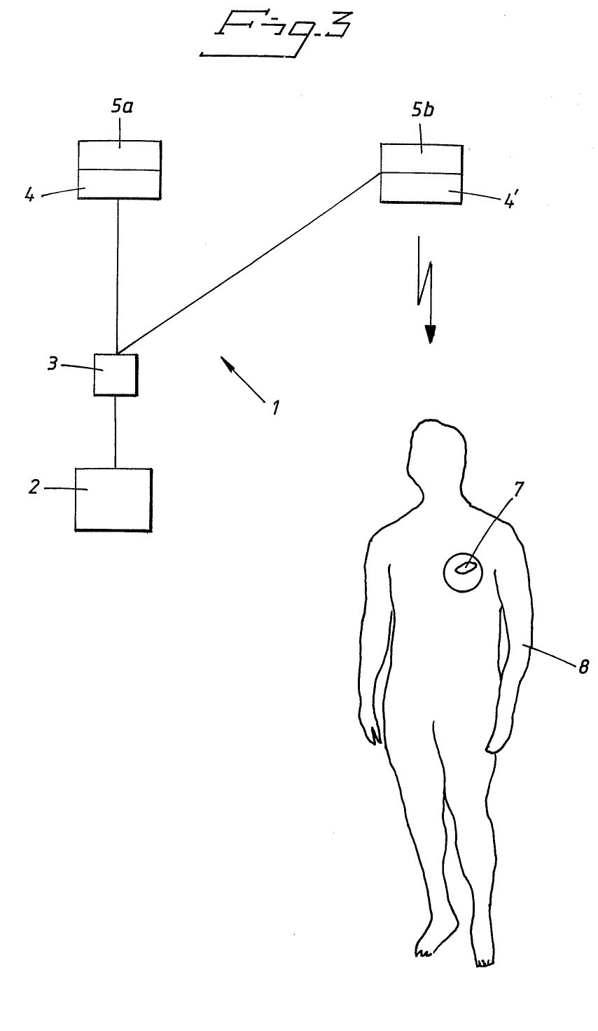

In FIG. 1 a single transceiver unit 4 is shown, but in an alternative embodiment, shown in FIG. 2, several transceiver units 4, 4′, 4″ could be used. In fact, each antenna device 5a, 5b, . . . , 5n could include one or more antenna elements and one transceiver unit 4, 4′, 4″. The antenna devices 5a, 5b, . . . , 5n and their respective transceiver units 4, 4′, 4″ could form an integrated unit, as shown in FIG. 2, or be separated units. Utilizing several transceiver units enables the use of an ad-hoc structure, i.e. the antenna devices 5a, 5b, . . . 5n, embodying antenna elements and a transceiver unit, constitutes autonomous nodes, thereby providing increased robustness in the communication.

In the embodiment of FIG. 2, there is no need for a switch device 6, since each antenna device 5a, 5b, . . . , 5n comprises a transceiver unit 4. In other respects, the embodiment of FIG. 2 is similar to the embodiment in FIG. 1.

The number of antenna devices 5a, 5b, . . . , 5n may be different in different rooms, in dependence of the particular need in a certain room. For example, an exercise room used for monitoring the heart of a patient when subject to an increased heart rate, may be provided with a larger number of antennas, thereby increasing the spatial diversity and enabling the patient to freely move around within the room without risking a communication failure due to fading. In a smaller room, in which the patient is not moving around, it may suffice to use a single antenna device 5a, 5b, . . . , 5n.

In accordance with the invention, the placing of the antenna devices 5a, 5b, . . . , 5n may be optimized with regard to, on the one hand, the most probable placement of the patient in a room. As was mentioned above, the most probable location of the patient in a room may be readily determined for example in an x-ray examination room, in which the patient presumably is monitored when being in situ for being x-rayed. The antennas may be mounted on the walls, the ceiling or even within equipment such as x-ray equipment or a hospital bed, or in a hospital room, such as a waiting room or an operating room. Thereby it is easy to optimize the communication between the patient-related device and the antennas of the medical apparatus in advance. In addition, when positioning the antenna devices 5a, 5b, . . . , 5n one should also consider near-field interference, and in particular their mutual coupling. Mutual coupling is pronounced up to a few wavelengths, and requires the space between adjacent antennas to be no less than a half-wavelength, the distance thus depending on the frequency in question. The signal at antenna device locations spaced a few wavelengths apart are almost independent, so increasing the distance between antennas would be beneficial. In accordance with the state of the art, the antennas are mounted to the programmer, whereby the distance between the antennas is limited to the size of the programmer. In contrast to this, the programmer 2 in accordance with the invention may be made portable, and in particular hand-held. Since the antenna devices 5a, 5b, . . . , 5n are not physically part of the casing containing the programmer 2, i.e. not in physical contact with the programmer 2, there are no restrictions being placed on the size of the programmer 2 for accommodating a plurality of antennas. Therefore the size of the programmer may be reduced considerably, and a user may easily bring the programmer 2 along if desired. In particular, the antenna devices 5a, 5b, . . . , 5n may be placed at locations such that the distance between them is larger than the largest external length of the programmer, and also such that the antenna devices 5a, 5b, . . . , 5n are separated at least two wavelengths apart in order to achieve appropriate spatial diversity. However, it is to be understood that the programmer 2 may, in an alternative embodiment, have a state-of-the art size and be arranged on a movable rack such as a roller table or the like.

In the prior art referred to in the introductory part of the description, the distance between the patient and the programmer is critical. In fact, as soon as the programmer, which includes antennas permanently mounted to it, is moved relative the patient the signal reception has to be assessed once more. In accordance with the invention, there is no longer a need for such tedious optimization.

Although the medical apparatus in accordance with the invention has been described above utilizing antenna devices separated from the programmer, it does not exclude the additional use of antennas mounted to the programmer.

FIG. 3 shows a system in accordance with the present invention. A medical apparatus 1 in accordance with the invention, comprising a programmer 2, a control unit 3, and antenna devices 5a, 5b, . . . , 5n, with radio transceiver units 4, 4′ . . . are utilized for monitoring and/or transmitting programming instructions to a patient related device 7, here shown to be an implantable device, implanted into a patient 8. The implantable medical device 7 has a radio transceiver enabled for communication with the medical apparatus 1 of the present invention.

Although modifications and changes may be suggested by those skilled in the art, it is the intention of the inventors to embody within the patent warranted heron all changes and modifications as reasonably and properly come within the scope of their contribution to the art.

1-24. (canceled)

25. A medical system comprising:
an implantable medical device configured for implantation in a living subject to interact with the subject,
an extracorporeal device comprising a processor that processes information selected from the group consisting of programming instructions for said implantable medical device and monitoring data received from said implantable medical device;
at least two antenna devices located at respectively different locations, said implantable medical device communicating with at least one of said two antenna devices;
said extracorporeal device also communicating with said at least two antenna devices to exchange said information with said implantable medical device via at least one of said two antenna devices; and
said at least two antenna devices being physically separated from said extracorporeal device and said extracorporeal device communicating with each of said at least two antenna devices via a communication link that allows said extracorporeal device to be freely moved relative to said at least two antenna devices, said communication link being selected from the group consisting of hard-wired communication links and wireless communication links.

26. A medical system as claimed in claim 25 comprising a control unit that measures a signal quality parameter of signals communicated from said implantable medical device to each of said at least two antenna devices, and that selects one of said at least two antenna devices for subsequent reception of signals from said implantable medical device dependent on the measured signal quality parameter.

27. A medical system as claimed in claim 26 wherein said control unit selects one of said at least two antenna devices for communicating signals to said implantable medical device dependent on said measured signal quality parameter.

28. A medical system as claimed in claim 26 wherein said implantable medical device includes, in signals communicated to at least one of said at least two antenna devices, a measure of a signal quality parameter of signals previously communicated to the implantable medical device that have been distorted by a transmission medium.

29. A medical system as claimed in claim 26 wherein said control unit measures a parameter, as said signal quality parameter, selected from the group consisting of RSSI, BER, and C/N ratio.

30. A medical system as claimed in claim 26 wherein said control unit measures said signal quality parameter in a measurement protocol selected from the group consisting of regular measurements and a continuous measurement.

31. A medical system as claimed in claim 26 wherein said control unit is connected between said extracorporeal device and said at least two antenna devices.

32. A medical system as claimed in claim 26 wherein said control unit is physically separate from said extracorporeal unit and communicates with said extracorporeal antenna unit via a first communication link selected from the group consisting of hard-wired communication links and wireless communication links, and communicates with each of said at least two antenna devices via a second communication link selected from the group consisting of hard-wired communication links and wireless communication links.

33. A medical system as claimed in claim 26 wherein said control unit is integrated into said extracorporeal device, and communicates with each of said at least two antenna devices via a communication link selected from the group consisting of hard-wired communication links and wireless communication links.

34. A medical system as claimed in claim 26 wherein said control unit is integrated into one of said at least two antenna devices, and communicates with said extracorporeal device via a communication link selected from the group consisting of hard-wired communication links and wireless communication links.

35. A medical system as claimed in claim 26 wherein said control unit comprises radio-based circuitry that transmits signals to and receives signals from said implantable medical device.

36. A medical system as claimed in claim 25 wherein each of said at least two antenna devices comprises at least one amplifier.

37. A medical system as claimed in claim 25 wherein each of said at least two antenna devices comprises a radio transceiver unit.

38. A medical system as claimed in claim 25 wherein each of said antenna devices comprises a mount that fixedly mounts that antenna device.

39. A medical system as claimed in claim 38 wherein said mount is a mount selected from the group consisting of sealing mounts and wall mounts.

40. A medical system as claimed in claim 25 wherein each of said antenna devices comprises a conductive radiating antenna element, the respective conductive radiating antenna elements of said at least two antenna devices both emitting and receiving radio waves having substantially parallel polarization, providing spatial diversity independently of polarization diversity.

41. A medical system as claimed in claim 25 wherein each of said antenna devices comprises at least one conductive radiating antenna element, with the antenna element of one of said at least two antenna devices emitting and receiving radio waves having a polarization that is orthogonal to radio waves emitted and received by the antenna element of another of said at least two antenna devices.

42. A medical system as claimed in claim 41 comprising a control unit that measures a signal quality parameters of signals received from said implantable medical device respectively by the antenna elements of the respective antenna devices, and selects one of said antenna elements in one of said antenna devices for subsequent communication of signals with said implantable medical device dependent on said measured signal quality parameter.

43. A medical system as claimed in claim 25 wherein said extracorporeal device is portable

44. A medical system as claimed in claim 43 wherein said extracorporeal device is a handheld device.

45. A medical system as claimed in claim 25 comprising a mounting unit to which said extracorporeal device is mounted, said mounting unit being selected from the group consisting of a moveable rack and a roller cart.

46. A medical system as claimed in claim 25 wherein said extracorporeal device comprises a keyboard communicating with said processor to allow manual inputs to be made to said processor, and a display unit connected to said processor to allow visual display of at least some of said information.

47. A medical system as claimed in claim 25 wherein said extracorporeal device has a device length, and wherein said at least two antenna devices are located at said respectively different locations at respective distances from said extracorporeal device that are larger than said device length.
